(19) United States
(12) Patent Application Publication
(10) Pub. No.: US 2016/0274104 A1
(43) Pub. Date: Sep. 22, 2016

(54) TEST METHOD FOR DETERMINGING BIOMARKERS

(71) Applicant: ANITEST OY, Kuopio (FI)

(72) Inventors: Erkki AMINOFF, Kuopio (FI); Jukka HALLMAN, Kuopio (FI); Jari HUUSKONEN, Kuopio (FI); Sami KOSKIMÄKI, Kuopio (FI); Anssi KUUTTI, Kuopio (FI); Jouko LUUKARINEN, Kuopio (FI); Mikko PITKÄNEN, Kuopio (FI)

(21) Appl. No.: 14/911,882

(22) PCT Filed: Aug. 12, 2014
(86) PCT No.: PCT/EP2014/067234

§ 371 Date: Feb. 12, 2016

(30) Foreign Application Priority Data
Aug. 13, 2013 (FI) 20135827

(51) Int. Cl. G01N 33/558; G01N 33/74; G06K 9/66; G06K 9/00; G06T 7/00

(52) U.S. Cl. CPC G01N 33/558; G06K 9/00127; G06T 7/0012; G06K 9/66; G01N 33/743; G06T 2207/20081

(57) ABSTRACT

The present invention relates to the field of human and veterinary biomarker tests and more particularly to test kits and methods for determining a result based on the presence, absence or concentration of a biomarker or biomarkers from a sample of a subject. More particularly the present invention relates to a test arrangement for determining the presence, absence or concentration of a biomarker. Also use of a combination of a test and a mobile device executable application for determining the presence, absence or concentration of a biomarker is within the scope of the present invention.

### FIELD OF THE INVENTION

The present invention relates to the field of human and veterinary biomarker tests and more particularly to test kits and methods for determining a result based on the presence, absence or concentration of a biomarker or biomarkers from a sample of a subject. More particularly the present invention relates to a test arrangement for determining the presence, absence or concentration of a biomarker. Also use of a combination of a test and a mobile device executable application for determining the presence, absence or concentration of a biomarker is within the scope of the present invention.

### BACKGROUND OF THE INVENTION

Biomarkers of biological samples are usually identified in laboratories. However, easy and quick home tests available for anyone are also used for determining biomarkers from human samples. Pregnancy tests are a well-known example of these home tests present on market. Smartphones provide a basis for further developments of medical home tests. Recently an application called uChek urinalysis system has been developed for iPhone. The app is one of the first that turns the iPhone into a medical device. The application is designed to read urinalysis test strips that are normally examined by users and compared to a color-coded chart or by dedicated reading devices. With the uChek system, people can take a picture of the strip with the iPhone's camera and then receive an automated readout of parameters like glucose, urobilinogen, pH, ketone and more. The app also stores results which then can be analyzed over time.

However, more developed methods and means for detecting the presence, absence or concentrations of biomarkers are needed. Simpler, more user-friendly, more cost effective and quicker tests are needed for example for home use.

### BRIEF DESCRIPTION OF THE INVENTION

An object of the present invention is to provide methods and tools for responding to the need of more developed, easy-to-use tests for determining various biomarkers.

The invention is based on the idea of providing a novel mobile device executable application, which helps the user in analyzing the results of tests. This helps to classify or detect the physiological status of the subject.

An advantage is that a user can easily purchase a biomarker test, simply use it at home, take one or more images of the test by a smartphone and get the results of the biomarker test and possibly also instructions for further actions from the smartphone. The results of the presence, absence or concentration of a biomarker in a sample are very quickly available for a user after applying the sample to the test. The user may easily download the application for reading the biomarker test results.

The invention relates to a method, a use, a mobile device and an arrangement defined in the independent claims. Different embodiments of the invention are disclosed in the dependent claims.

An aspect relates to a test method for determining a result based on the presence, absence or concentration of a biomarker in a sample of a subject, wherein the method comprises the following steps

a) contacting a sample obtained from the subject with a test for determining a biomarker or biomarkers,

b) allowing the sample to react in the test, and

c) capturing one or more images of the reaction results and the control in the test,

d) inputting the at least one image to an image processing, the image processing outputting one or more test results indicating the presence, absence or concentration of the biomarker in the sample, and

e) showing the test results and/or a conclusion drawn from the test results via a graphical user interface.

Also, an aspect relates to the use of a combination of a test and a mobile device configured to determine the presence, absence or concentration of a biomarker in a sample of a subject from an image of the used test.

Still, an aspect relates to a test arrangement for determining the presence, absence or concentration of a biomarker in a sample of a subject, comprising

a) a test for determining biomarkers, and

b) a mobile device configured to determine the presence, absence or concentration of a biomarker in a sample of a subject from one or more images of the used test.

Furthermore, one aspect relates to a mobile device comprising:

at least one user interface;

at least one camera unit;

at least one processor and at least one memory including a computer program code, wherein the at least one memory and the computer program code are configured, with the at least one processor, to cause the mobile device to implement at least an analyzer tool loaded into the mobile device and to perform, in response to detecting that the analyzer tool is selected via the user interface, operations comprising:

activating the at least one camera unit for taking one or more images;

inputting the one or more images to an image processing of the analyzer tool, the image processing being configured to determine image by image from one image a grey level of a first background area in a test, a grey level of a second background area in the test, a grey level of a first line splitting the first background area and a grey level of a second line splitting the second background area;

inputting the grey levels obtained as output from the image processing to a trained neural network of the analyzer tool, the neural network being trained to output the presence, absence or concentration of a biomarker;

outputting via the user interface the output of the trained neural network and/or a conclusion determined from the output of the trained neural network.

### BRIEF DESCRIPTION OF THE DRAWINGS

In the following, exemplary embodiments will be described in greater detail with reference to accompanying drawings, in which

FIG. 1 shows the principle of the lateral flow assay;

FIG. 2A shows a simplified block diagram of a mobile device according to an exemplary embodiment;

FIG. 2B shows simplified architecture of a system and block diagrams of some apparatuses according to another exemplary embodiment;

FIG. 3 shows an image and what is defined from the image;

FIGS. 4 to 7 are flow charts illustrating different exemplary functionalities; and

FIGS. 8 and 9 are block diagrams of exemplary apparatuses.

### DETAILED DESCRIPTION OF SOME EMBODIMENTS

The following embodiments are exemplary. Although the specification may refer to “an”, “one”, or “some” embodiment(s) in several locations, this does not necessarily mean that each such reference is to the same embodiment(s), or that the feature only applies to a single embodiment. Single features of different embodiments may also be combined to provide other embodiments.

### Subjects and Samples

The method, test or test arrangement of the invention is suitable for any subject in need of determining the presence, absence or concentration of a biomarker from a sample obtained from the body. The subject may be any healthy person or any person suffering from or suspected of suffering from mild, moderate or severe symptoms. In one embodiment of the invention, the subject is a human or an animal.

In one embodiment of the invention, the animal is a canine, feline, equine, pig, ruminant, camelid or zoo animal. As used herein “canine” refers to the family Canidae of carnivorous and omnivorous mammals that includes domestic dogs, wolves, foxes, jackals, coyotes, and other dog-like mammals. “Feline” refers to family Felidae including the domestic cat as well as all wild cats such as the tiger, the lion, the jaguar, the leopard, the cougar, the cheetah, the lynxes and the ocelot. “Equine” refers to any member of the genus Equus, including any horse. Equus belongs to the family Equidae including horses, donkeys, and zebras. As used herein “ruminant” refers to an animal which has a four-compartment stomach and chews the feed over again such as cows, goats, sheep, llamas or camelids. As used herein “a zoo animal” refers to an animal which lives in a zoo, such as a monkey, chimpanzee, gorilla, canine, feline, equine, pig, ruminant, camelid, llama, any bird, any lizard or any water animal. More preferably the animal is selected from a group consisting of domestic animals (such as a dog or a cat), zoo animals (such as a monkey) or livestock and production animals (such as a cow, a horse or a pig). Most preferably the animal is selected from a group consisting of a dog, a cat, a cow, a horse and a pig.

At home any sample which is easily provided can be utilized for the present invention. Depending on the test used, for example urine or saliva samples are user-friendly obtained from a subject. The sample may be selected from a group consisting of a tissue fragment, a secretion sample, a blood sample and another suitable sample. As used herein “a secretion sample” refers to a saliva, urine, feces, breathing or brush sample. In one embodiment of the invention the sample is a blood, saliva, feces or urine sample. As used herein “a blood sample” refers to any normal blood sample or any part or further application of it. Therefore, the blood sample may for example be in the form of whole blood, serum or plasma. Most preferably, the sample is a urine sample.

A sample can be either in a solid or liquid form, preferably as a fluid. Amount of a sample needed for a biomarker test varies depending on a test used and a sample collected, but a droplet may be enough for some tests and some milliliters or centiliters of a sample may be needed for other tests. Samples may be pre-treated before use for the biomarker test, for example by making a solid sample to a liquid form or by extracting proteins or DNA/RNA from a sample. However, the most suitable samples do not need any pre-treatments and are applied as untreated samples directly to the test strip.

### Tests

The present invention utilizes ready-to-use home tests. As used herein “a test” refers to any biomarker test that can be fast and easily used at home. A sample of an individual for the test can also be taken at home. Results of a test can be achieved for example within 45, 30, 20, 15 or 10 minutes, or even within 5, 4, 3 or 2 minutes from contacting a sample to the test. A biomarker test refers to any test, which determines the presence, absence or concentration of a biomarker in a sample obtained from a subject.

Even though any person can use the test at home, also professionals may exploit it in clinics, hospitals or ambulances as well as in laboratories. The test may be a POC test. As used herein “POC testing” refers to a medical testing at or near the site of patient care.

A test of the present invention may be in any form suitable for home use. For example the test may be in the form of a strip, such as made of paper or plastic. Test pads of a strip change visually, when contacted with the sample. Any visual changes such as a change of the color, intensity or lightness can be used for detecting the results of a test. In addition to test strips, also other forms of tests, like test sticks, can be used in the present invention.

In one embodiment of the invention the test is a DNA test. In another embodiment of the invention the test is a conventional color strip test for example as described by Leuvering J H W et al. (J Immunoassay Immunochem (1980) 1:77-91), Leuvering J H W et al. (J Immunol Methods (1981) 45:183-194), van Amerongen A et al. (J Biotechnol (1993) 30:185-195), Osikowicz G et al. (Clin Chem (1990) 36:1586), or Posthuma-Trumpie G et al. (Anal Bioanal Chem (2009) 393:569-582).

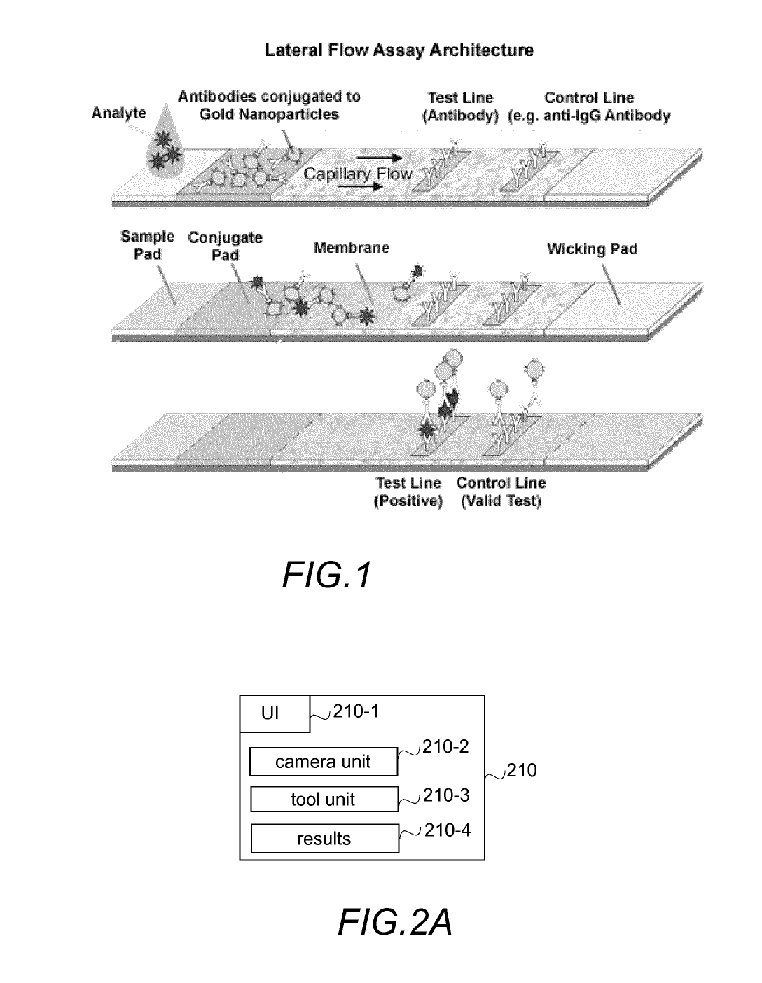

In one embodiment of the invention the test is a lateral flow assay. Lateral flow assays are simple devices intended to detect the presence (or absence or amount) of a target analyte in a sample without the need for specialized and costly equipment. The technology is based on a series of capillary beds, such as pieces of porous paper or sintered polymer. Each of these elements has the capacity to transport fluid spontaneously. The fluid migrates to the element with the so-called conjugate for an optimized chemical reaction between the target molecule (e.g., an antigen) and its chemical partner (e.g., antibody) that has been immobilized on the particle's surface. In one combined transport action the sample and conjugate mix while flowing through the porous structure. In this way, the analyte binds to the particles while migrating further through the capillary bed. By the time the sample-conjugate mix reaches the strips where a third “capture” molecule has been immobilized, analyte has been bound on the particle and the third “capture” molecule binds the complex. After more fluid has passed the stripes, particles accumulate and the stripe-area changes visually. Typically there are at least two stripes: one (the control) that captures any particle and thereby shows that reaction conditions and technology worked fine, the second contains a specific capture molecule and only captures those particles onto which an analyte molecule has been immobilized. Finally the fluid enters the final porous material, a waste container. Lateral Flow Tests can operate as either competitive or sandwich assays. (see FIG. 1)

In a specific embodiment of the invention the test comprises an antibody based assay.

For the test of the invention only one sample from an individual is needed. Alternatively, two or more samples from one or more individuals can be applied to a test. Optionally, also an internal positive and/or negative control may be comprised in the test. The quick test kit or arrangement may further comprise any conventionally used reagents which are well known among the persons skilled in the art. The test kit or test arrangement may also comprise instructions for using the test or the combination of a test and a mobile device. The methods, kits and arrangements of the present invention provide quantitative, semi-quantitative or qualitative measuring of the biomarkers in a biological sample. In the present in vitro-tests the presence, absence, amount or aberrant concentration of a biomarker is identified.

As used herein “reaction results” refers to results of the test shown by visible changes of the test (e.g. stripes). As used herein “test results” refers to results indicating the presence, absence or concentration of a biomarker or biomarkers. The test results may be given by the mobile device for example in the form of exact biomarker amounts or concentrations or in the form of a low or high amount or concentration of a biomarker compared to a normal level, or the presence or absence of a biomarker.

### Biomarkers

The present invention helps in detecting one or more biomarkers from a biological sample. As used herein “the presence or absence of a biomarker” refers to the presence of a biomarker in any amount or concentration, or absence of a biomarker. As used herein “a result based on the presence, absence or concentration of a biomarker” refers to test results and/or to any conclusion drawn from the test results (e.g. certain concentration of progesterone in a biological sample of a dog refers to ovulation).

The present invention utilizes a test arrangement comprising a biomarker test and a mobile device and is able to detect biomarkers from a sample in a concentration of at least 50 nmol/l or at least 100 nmol/l, specifically 50-2000 nmol/l, and more specifically 100-1000 nmol/l. The prior art home tests have not been able to detect as low concentrations of biomarkers as the present invention. Also, by the test arrangement and method of the invention it is possible to get very reliable and accurate results at home. The present test arrangement reaches accuracy of ±10% in biomarker concentrations, this accuracy being as good as by the laboratory methods (e.g. analyzer Siemens Immulite 2000).

The most important aim of the present invention is to give knowledge of the health or welfare of a subject. Any test results showing deviations from the normal may embolden a subject to change a way of life e.g. to control the amount of food or sugar or to rest more. On the other hand test results showing deviations from the normal may guide a subject to the doctor. As used herein, a deviation includes any deviation, not only significant deviation from the normal. In one embodiment of the invention a deviation includes only significant deviation from the normal. “Significant deviation” refers to a deviation from normal values shown by a statistical test with p-value equal or less than 0.5. Thus, the test of the invention serves as a screening tool for detecting any health aberrations.

Biomarkers, also called as biological markers, are indicators of biological states. Biomarkers are objectively measured and evaluated as indicators of for example normal biological processes, pathogenic processes, or pharmacologic responses to a therapeutic intervention. Biomarker is a substance whose presence, absence, aberrant concentration or aberrant activity indicates a particular state. Most specifically, the present invention identifies the presence/absence or concentration of one or more biomarkers. The test of the invention may identify for example one, two, three, four, five, six, seven, eight, nine, ten or even more biomarkers.

For example biomarkers can be any molecules such as proteins, antibodies, lipids or metabolites and furthermore DNA, RNA or amino acid sequences, or any combinations thereof. In a specific embodiment of the invention, the biomarker is selected from a group consisting of cortisol, RBP (Retinol Binding Protein), bile acids, progesterone, BNP (B-type Natriuretic Peptide or Brain-derived Natriuretic Peptide), proBNP, NT-proBNP, troponin I (TnI), troponin T (TnT), DHEA (DiHydroEpiAndrosteron), DHEA-S (DiHydroEpiAndrosteroni-Sulphate), PSA (Prostata Specific Antigen), PAP (Prostatic Acid Phosphatase), trypsinogen, myoglobin, rheumatoid factor, cyclic citrullinated peptide, neopterin, catecholamines, deoxypyridinoline, N-telopeptide (NTX), beta-2-microglobulin.

Cortisol has been associated with a stress related condition, RBP (Retinol Binding Protein) with dysfunctions of a kidney, bile acids with dysfunctions of a liver, progesterone with pregnancy, BNP (B-type Natriuretic Peptide or Brain-derived Natriuretic Peptide) proBNP or NT-proBNP with heart dysfunctions or heart defects, troponin I (TnI) or troponin T (TnT) with heart muscle damages, DHEA (DiHydroEpiAndrosteron) or DHEA-S (DiHydroEpiAndrosteroni-Sulphate) with a stress related condition or overweight, PSA (Prostata Specific Antigen) or PAP (Prostatic Acid Phosphatase) with prostate tumors, trypsinogen with pancreatitis, myoglobin with heart or skeletal muscle damage, rheumatoid factor, cyclic citrullinated peptide or neopterin with autoimmune/rheumatoid diseases, catecholamines with stress or with foechromocytoma, deoxypyridinoline with bone/teeth metabolism, N-telopeptide with bone metabolism and beta-2-microglobulin with different forms of cancer.

Indeed, any biomarkers which have been associated with disorders may also be detected by the present invention. These disorders include at least urinary tract infections, stress related conditions, dysfunction of a kidney or liver, hepatitis, anemia, metabolic acidosis and alkalosis, respiratory acidosis and alkalosis, diabetes mellitus, diabetes ketoacidosis, diabetes insipidus, diarrhea, starvation, biliary tract infections, pregnancy, dehydration, heart dysfunction or heart defect, heart muscle damage, pancreatitis, menstruation and cancers. In one embodiment of the invention the disorder is selected from a group consisting of a stress related condition, loss of weight, dysfunction of a kidney or liver, pregnancy, heart dysfunction or heart defect, heart muscle damage, skeletal muscle damage (e.g. rhabdomyolysis), skeletal muscle dysfunction, dystrophy or other skeletal muscle disorder, cancer and pancreatitis. “A stress related condition” refers to a condition resulting in physical or mental stress in either acute or chronic manner. Stress-related medical conditions include but are not limited to gastrointestinal, cardiovascular, respiratory, musculoskeletal, skin, psychological or reproductive disorders.

Dysfunctions of a kidney or liver include at least cirrhosis of the liver, renal calculi, nephropathy, nephritis and any other condition affecting the kidneys to function abnormally. Heart dysfunctions or heart defects include at least heart failures, congestive heart failures and atrial fibrillation. In one specific embodiment of the invention the quick test is an antibody based test (e.g. lateral flow test) for an animal, determining aberrant cortisol concentration in a urine sample obtained from the animal. In a specific embodiment of the invention, cortisol concentrations ranging from 100 nmol/l to 1000 nmol/l can be determined. In another specific embodiment of the invention smart phone application is coded to give a result “stress level low” when the cortisol concentration is less than 350 nmol/l, “stress level medium” when the concentration is between 350-700 nmol/l, and “stress level high” when the concentration is more than 700 nmol/l.

### Analyzer Tool

Most semi-automated biological sample analyzer machines may use reflectance based methods and specialized hardware and software to measure, process and report results from reagent strips. For example, the uChek urine analyzer has the same working principle and is substantially equivalent to most such machines. The uChek system makes use of the image sensor, software and hardware on a smartphone, and, in conjunction with the colormat and cuboid from the kit, is able to perform the same function as most commercially available semi-automated urine analyzer machines.

In the present invention the analyzer tool may be provided as a stand-alone tool, for example as an application (app) downloadable to a mobile device or as a distributed tool comprising for example a centralized analyzing application and an application (ap) downloadable to a mobile device, the application being configured to send one or more captured images to the centralized analyzing application, receive a corresponding result and to output it to a user.

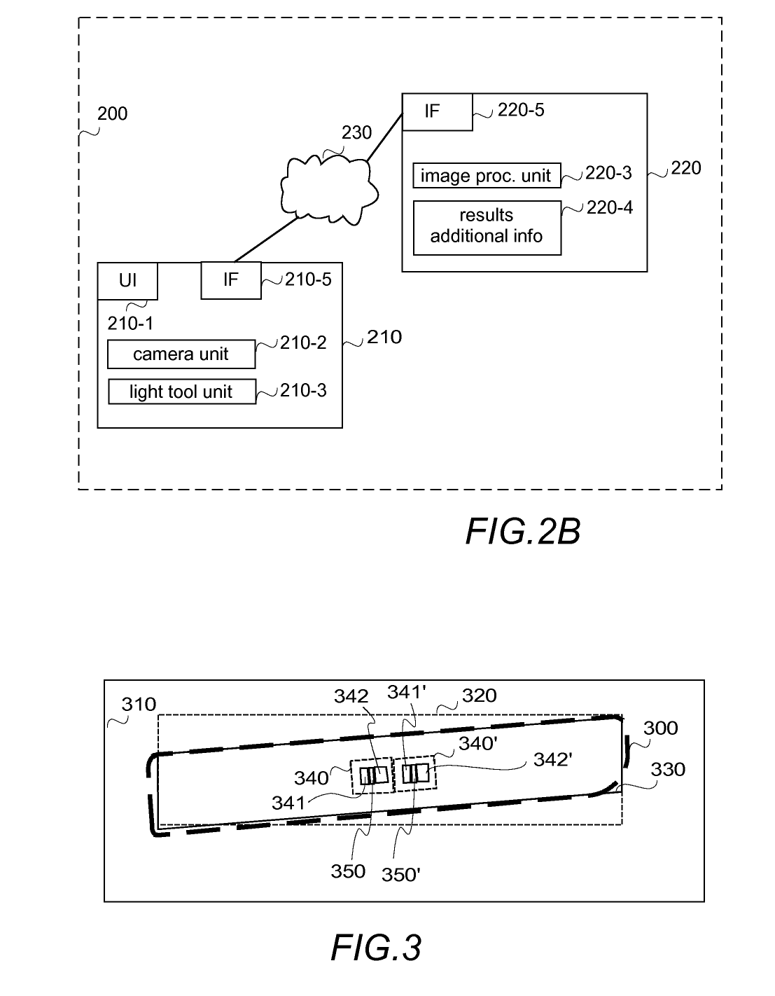

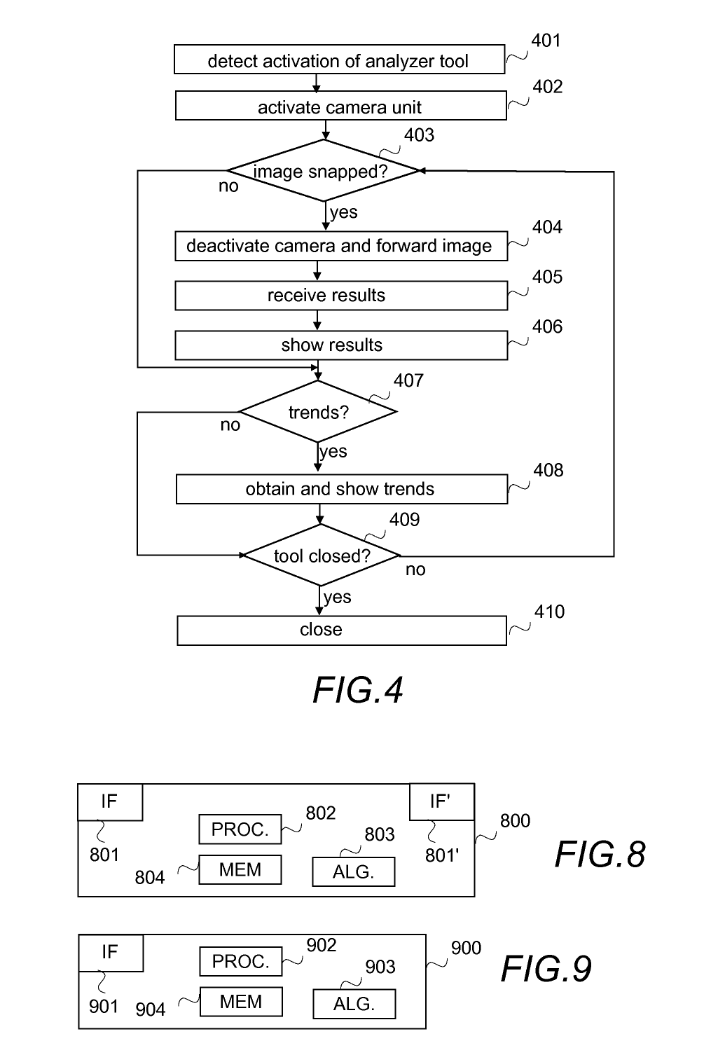

FIG. 2A is a simplified block chart illustrating an exemplary embodiment of a mobile device 210 in which the analyzer tool is a standalone tool, i.e. a tool that does not necessarily require a network connection to function. For the analyzer tool, the mobile device comprises one or more user interfaces 210-1 for starting the analyzer tool and for outputting results, a camera unit 210-2 for capturing images, a tool unit for image processing to obtain the results, and in the illustrated example a memory 210-4 for storing results. The stored results may be used for different statistics, like generating a time series to find out one or more trend.

The mobile device 210 refers to a computing device (equipment). Such computing devices (apparatuses) include wireless mobile communication devices operating with or without a subscriber identification module in hardware or in software, including, but not limited to, the following types of devices: smart-phone, personal digital assistant (PDA), tablet, etc. Further, the tool unit may be built to operate on any mobile operating system, like iOS, Meego, Sailfish, Windows, Android, etc.

FIG. 2B shows simplified architecture of a system and block diagrams of some apparatuses according to another exemplary embodiment in which the analyzer tool is a distributed tool requiring a network connection to function. In the illustrated example of FIG. 2B the system 200 comprises one or more mobile devices 210′ (only one is illustrated in FIG. 2B) connectable through one or more networks 230 to a server apparatus 220.

As said above, the mobile device refers to a computing device (equipment). In the illustrated example of FIG. 2B, the mobile device 210′ comprises for the analyzer tool one or more user interfaces 210-1 for starting the analyzer tool and for outputting results, a camera unit 210-2 for capturing images, a light tool unit 210-3′ at least for conveying images and results, and one or more interfaces 210-5 for establishing a network connection and for data exchange with the server apparatus. Since the light tool unit 210-3′ is configured to provide less functionalities than the tool unit in the stand-alone implementation, the mobile device 210′ may be a simpler computing device than the mobile device in the example of FIG. 2A, i.e. it does not need to have as much computational capacity. For example, in addition to the above listed examples, the mobile device may be a feature phone or a digital camera with a wireless access and some inbuilt processing capacity.

The server apparatus 220 refers to a computing device (equipment) configured to perform the analyzing task on behalf of the mobile devices. For that purpose the server apparatus 220 comprises an interface 220-5 for exchanging data with the mobile devices, an image processing unit 220-3 for processing images and outputting one or more results, and in the example for associating the results with additional information, and one or more memories 220-4 for storing the results at least client specifically and for storing the additional information. The additional information may comprise for a specific result at least one of the following: a description on a possible problem and causes, “home tricks” to alleviate the problem, instructions to turn to vet/physician for medical treatment, and one or more hyperlinks via which more information is obtainable.

An example of a server apparatus is a computer configured for specific purpose to provide one or more specific services.

A network through which the server apparatus and the mobile device may be connected to each other, may be any kind of a network or a direct connection, or a combination of a direct connection and one or more networks, or the connection may be over two or more networks, which may be of different type. Examples include a bluetooth connection, a wireless local area network, different mobile networks (3GPP, LTE and beyound, IMT, etc.) and Internet.

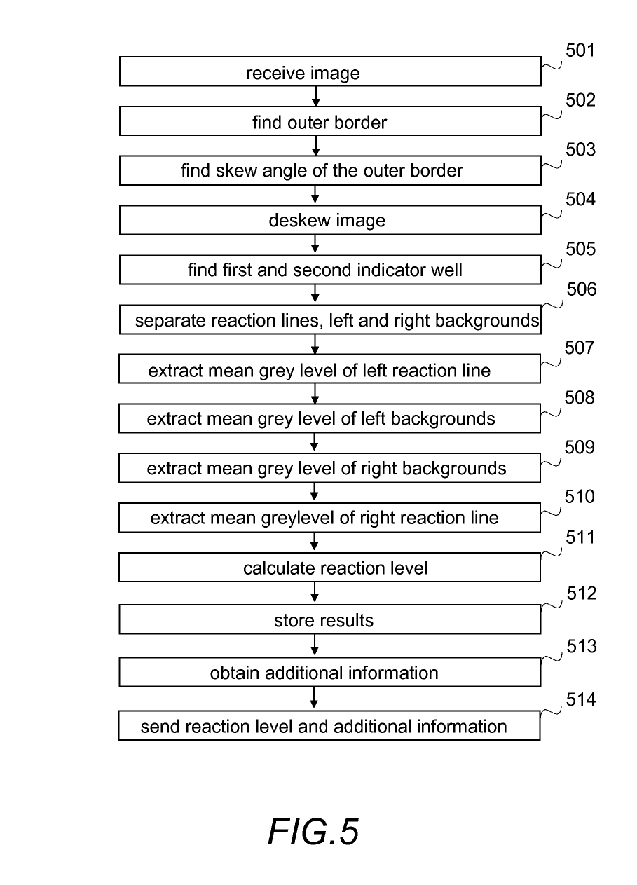

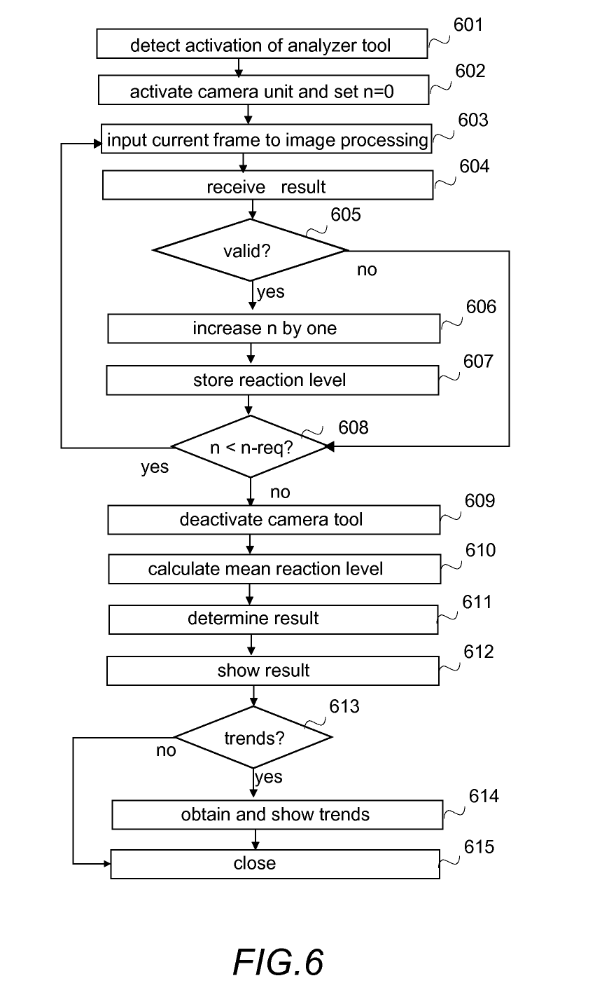

FIG. 3 illustrates an image 310 of the test 300 and what is search for in the image during image processing. The image 310 is captured in a process described with FIG. 4 or with FIG. 6 and processed in a process described with FIG. 5 or with FIG. 7. It should be appreciated that although in FIG. 3 the image 310 is taken in such a way that the whole test 300 is inside the image 310 that need not to be the case; it suffices that wells and preferably but not necessarily, part of the outer border of the test, are within the image. Further, it should be appreciated that term “well” as used herein covers any visible area/place on a test (or in a test), like a pad on a test strip, which is intended to contact or react with the sample.

FIG. 4 is a flow chart illustrating an exemplary functionality of the light tool unit. In the illustrated example it is assumed that in addition to the result the application may also provide trends, such as three or more last results. It should be appreciated that the application may be configured to provide any information relating to the analysed features. The information may be based on historical results of the animal in question, historical results of corresponding animals, etc. Further, in the example of FIG. 4 it is assumed, for the sake of clarity, that the analyzer tool is used for measurements of one sample of one individual for one purpose without restricting the example and corresponding implementations to such a solution. For one skilled in the art it is obvious how to apply the described functionality to two or more samples and/or to two or more purposes and associate and handle results and trends person/pet-specifically.

Referring to FIG. 4, when the tool unit detects in step 401 that a user has activated the analyzer tool, for example by clicking a corresponding icon in a graphical user interface of the mobile device, the camera unit is activated in step 402. Depending on an implementation, the camera unit may be activated in response to the user selecting a specific icon or text, for example, like “analyze”, when the user is navigating within the analyser tool, or in response to the analyzer tool being activated, or in response to the activated analyzer tool prompting the user to select amongst different use options of the tool. Then it is monitored in step 403 whether or not an image is snapped, i.e. captured, and if not, whether or not the user selects to request trends (step 407) and if not, whether or not the user closes the analyzer tool (in step 409). These monitoring steps are repeated until a user selection of one of the monitored steps is selected.

If an image is snapped (step 403), the camera unit is deactivated in step 404 and the image is forwarded in step 404 for image processing, i.e. in the illustrated example to the server apparatus. Then it is waited few seconds until results are received in step 405. The received results, and possible additional information received with the results, are shown to the user via the user interface in step 406. Then the process proceeds to step 407 to continue the monitoring and repeating steps 403, 409 and 407.

If trends is selected (step 407), the trends, like a time series of results, are obtained and shown in step 408. It should be appreciated that in another implementation the user is able to select which type of trends she/he is interested in, and then those trends are obtained and shown. Then the process proceeds to step 409 to continue the monitoring and repeating steps 403, 409 and 407.

If the tool is closed (step 409), the analyzer tool is closed in step 410.

It should be appreciated that if the results is associated with a hyperlink, and the hyperlink is clicked or otherwise selected, the hyperlink is followed by the mobile device by starting a browser application and outputting the content obtainable via the hyperlink to the user interface.

FIG. 5 is a flow chart illustrating an exemplary functionality of the image processing unit receiving the image from the light processing tool described above. In other words, it explains in more detail an exemplary image processing that outputs one or more results. In the illustrated example it is assumed that there may be three reaction levels. However, one skilled in the art may easily adapt the procedure to obtain more reaction levels.

Referring to FIGS. 5 and 3, when the image is received in step 501, an outer border 320 of the test 300 is search and found from the image 310. An advantage provided by finding (determining) the outer border is that the image processing may be focused within the outer border, i.e. within the test, other information in the image is not processed. This also makes the image processing computationally lighter and thereby faster. The outer border 320 is found by means of a statistical classifier, for example. An example of such a statistical classifier is a CascadeClassifier provided by openCV and supporting LBP (Local Binary Patterns) features. LBP features are integer, so both training and detection with LBP are fast one. Further, an advantage is that even an advanced mobile device comprises the computational resources needed by a trained CascadeClassifier with LBP. Preparation of training data (including positive data comprising thousands of images from the identifiable object in different positions in different lighting conditions, placed on different kinds of surfaces, etc. and negative data comprising thousands of images that do not contain the identifiable object) and the actual training of the statistical classifier are well known in the art and therefore need not be described in more detail here.

When the outer border is found, a skew angle of a box formed by the outer border is search for and found in step 503. The skew angle may be found by applying the Hough transform to outer border 320 to find out the location of the outer border 330 of the test and then determining the skew angle from the borders of 320 and 330. Then the image is deskewed in step 504 (not illustrated in FIG. 3) so that the image of the test, and hence the images of wells and a reaction line and a control line are straighten to facilitate the further analysis. For example, thanks to the deskewing, finding the wells and lines can be performed by searching for vertical and horizontal lines, which is computationally lighter procedure, i.e. needs and uses less computing resources, than searching for lines that may be in any angle.

After deskwewing, indicator wells, ore more precisely borders 340, 340′ defining corresponding boxes for the indicator wells are searched for and found in step 505 within the outer border 320 (outer box). The borders 340, 340′ are found by means of a statistical classifier, for example. The above described CascadeClassifier provided by openCV and supporting LBP (Local Binary Patterns) features may be used also herein, provided that the training data for the statistical classifier is different than the training data for the outer border.

Then the well boxes, i.e. the borders 340, 340′ are each separated in step 506 to a reaction line 350, 350′, a left background 341, 341′ and a right background 342, 342′. To find the reaction line area and even-colored backgrounds in a well, an adaptive thresholding and heuristic is applied to the area within the corresponding border 340, 340′. The adaptive thresholding may be an adaptive threshold function provided by openCV and intended to bring out, using a threshold value, pixels that are darker than most of the surrounding pixels. A split half method may be used to obtain the threshold value. For example a line in a black and white image is an adequate amount of black and white. However, these details are well known in the art, and therefore need not be described in more detail here. After the adaptive thresholding the borders and the reaction line should be in black, all the rest is white. The heuristic may be based on simple conclusions, like “if between two vertical black lines (i.e. vertical parts of the border 340 or 340′) a black line with width between x and y is found, it is determined to be the reaction line”.

When the backgrounds and reaction lines are found, the grey levels (values) of the boxes are obtainable. Using the grey levels and calculating a median of the grey levels, a mean grey level of the left reaction line 350 is extracted in step 507, a mean grey level of the left side boxes 341, 341′, i.e. left side backgrounds, is extracted in step 508, a mean grey level of the right side boxes 341, 341′, i.e. right side backgrounds, is extracted in step 509, and a mean grey level of the right reaction line 350′ is extracted in step 510. Depending on an implementation, the extraction may include also other functions like nonlinear filtering to filter noise and dirt, for example, and/or to determine whether or not the test is too dirty and/or have too many light reflections, i.e. bright spots, in the bottom of well, to be image processed.

Then the four mean grey levels are used to calculate a reaction level in step 511. The reaction level may be calculated by inputting the four mean grey levels as input data to a multilayer perceptron (MLP) neural network comprising one hidden layer with 2 to 15 neurons, for example with 6 neurons, and maps the input data onto a three outputs (classes), one for each reaction level, i.e. one for low reaction level, one for medium reaction level and one for high reaction level. Training data for the neural network comprises positive data for each class, i.e. in the illustrated example a positive data set for low reaction level, a positive data set for medium reaction level and a positive data set for high reaction level. A positive data set is received by repeating steps 1 to 510 for thousands of images, snapped from the identifiable object having the reaction level (class) for which the positive data set is collected, in different positions in different lighting conditions, placed on different kinds of surfaces, etc. The reason for using the neural network is that different cameras create different grey levels and a direct comparison between the different grey levels is not reliable enough, and the neural network overcomes the reliability issue and provides a “camera-independent” solution.

The reaction level is then stored in step 512, and associated information for the outputted reaction level is obtained from the memory in step 513, and then send in step 514 to the mobile device for outputting to the user.

In another exemplary embodiment, the image processing unit may be configured to send the reaction level to the light tool unit without performing steps of 512 and 513, and the light tool unit may be configured to store the results and possible obtain the additional information.

The stand-alone tool unit is configured to perform the steps in FIG. 4 and in FIG. 5 so that the information exchange is internal exchange. Further, when the steps are performed in the mobile device, the result are received (step 405) in praxis immediately after the image is captured (step 404).

FIGS. 6 and 7 are flow charts illustrating an exemplary functionality of another exemplary implementation of the updatable stand-alone tool unit, the functionality being divided just for illustrative purposes to image processing part (depicted in FIG. 7) and the other processing part (depicted in FIG. 6). Also in the illustrated example it is assumed that there may be three reaction levels. However, one skilled in the art may easily adapt the procedure to obtain more reaction levels. Also in the illustrated example it is assumed that in addition to the result the application may also provide trends, such as three or more last results. It should be appreciated that the application may be configured to provide any information relating to the analysed features, as described above. Further, also in the example of FIGS. 6 and 7 it is assumed, for the sake of clarity, that the analyzer tool is used for measurements of one sample of one individual for one purpose without restricting the example and corresponding implementations to such a solution. For one skilled in the art it is obvious how to apply the described functionality to two or more samples and/or to two or more purposes and associate and handle results and trends person/pet-specifically.

Referring to FIG. 6, when the tool unit detects in step 601 that a user has activated the analyzer tool, for example by clicking a corresponding icon in a graphical user interface of the mobile device, the camera unit is activated in step 602 to start to take a video from the test and the number n of the processed frames is set to be zero, and then a current frame is inputted in step 603 for image processing that is illustrated in FIG. 7.

Referring to FIGS. 7 and 3, when the image processing part receives in step 701 a frame, an outer border 320 (or at least part of the outer border) of the test 300 is search and found from the image 310 in step 702, as described above with FIG. 5, and the same means may be used as well herein. When the outer border is found, a skew angle of a box formed by the outer border is search for and found in step 703. and the frame is deskewed in step 704 (not illustrated in FIG. 3) so that the frame, and hence the images of wells and a reaction line and a control line are straighten to facilitate the further analysis, as described above with FIG. 5.

After deskwewing, indicator wells, ore more precisely borders 340, 340′ defining corresponding boxes for a first indicator well and a second indicator well, correspondingly, are searched for and found in step 705 within the outer border 320 (outer box). The borders 340, 340′ are found by means of a statistical classifier, for example, as described above with FIG. 5.

Then the well boxes, i.e. the borders 340, 340′ are each separated in step 706 to a reaction line 350, 350′, a left background 341, 341′ and a right background 342, 342′, for example as described above with FIG. 5.

When the backgrounds and reaction lines are found, the left back-ground 341 of the first well is combined in step 707 with the right back-ground 342 of the first well to form one combined area, called herein “first backgrounds”, and correspondingly the left background 341′ of the second well is combined in step 708 with the right background 342′ of the second well to form one combined area, called herein “second backgrounds”.

Then the “frame data” is ready to be analyzed and statistical information, like m points of k-quantile, of grey levels from the reaction line, control line and the combined areas are determined. (When k-quantiles are used, m is an integer that satisfies 0<m<k.) More precisely, m points of grey shade k-quantile of the left reaction line is extracted in point 709, m points of grey shade k-quantile of the combined area of the first backgrounds is extracted in point 710, m points of grey shade k-quantile of the combined area of the second backgrounds is extracted in point 711, and m points of grey shade k-quantile of the right reaction line is extracted in point 712. Depending on an implementation, the extraction may include also other functions like nonlinear filtering to filter noise and dirt, for example, and/or to determine whether or not the test is too dirty and/or have too many light reflections, i.e. bright spots, in the bottom of well, to be image processed.

Then the extracted grey levels are used to calculate (determine) a reaction level in step 713. The reaction level may be calculated by inputting the four mean grey levels as input data to a trained multilayer perceptron (MLP) neural network comprising one hidden layer with 2 to 15 neurons, for example with 6 neurons, that maps the input data onto a three outputs (classes), one for each reaction level, i.e. one for low reaction level, one for medium reaction level and one for high reaction level. The training of the neural network is described above with FIG. 5.

If the determination of the reaction level succeeds in step 713, the reaction level is determinable (step 714), and the reaction level is sent in step 715 as an output of the image processing to be further processed internally within the tool unit.

If the determination of the reaction level does not succeed, or any other step in the image processing fails, the reaction level is not determinable (step 714), and in the illustrated example an empty result is sent in step 716 as the output. It should be appreciated that any information that is clearly different from a reaction level may be sent instead.

Returning back to FIG. 6, when the reaction level is received in step 604, it is checked in step whether or not the result is a valid one, i.e. in the illustrated example, whether or not it contains a reaction level.

If the result is a valid one, the number n of the processed frames is increased by one in step 606, and the received reaction level, is stored in step 607.

In the illustrated example, a predefined amount of results is required, and hence it is then checked, in step 608, whether or not the number n of the processed frames is smaller than the amount n-reg of validly processed frames corresponding to the predefined amount of results. The amount n-req may be for example 1, 2, 4, 16, 32, 64, 102, 110, 113, etc. The bigger the amount n-req is the more accurate results are obtained but the more processing time is needed, so selection of the n-req depends on the biomarker, how many reaction levels are used, and what is the satisfactory accuracy, etc.

If the number n is smaller than the amount n-req (step 608), then the process proceeds to step 603, and a further frame is inputted to the image processing.

If the number n is not smaller than n-req (step 608), the camera unit is deactivated in step 609 and in the illustrated example a mean reaction level is calculated in step 610 from the stored reaction levels, and in step 611 the corresponding result is determined and shown in step 611 to the user. For example, the determining may comprise comparing the reaction level to limits. For example, the result may be one of the following depending on reaction level (concentration): “stress level low” when concentration is less than 350 nmol/l, “stress level medium” when the concentration is between 350 nmol/l to 700 nmol/l, and “stress level high” when the concentration is more than 700 nmol/l. However, it should be appreciated that in another implementation the mean reaction level may be outputted as such, in which case step 611 is omitted. Further, instead of the mean any other suitable statistical value, like average, may be used.

Further, it should be appreciated that in another example, after determining the result, additional information as described above with FIGS. 4 and 5, may be obtained and shown.

In the illustrated example, the result is shown to the user with a possibility to request trends in addition to the possibility to close.

If an input requesting trends is received (step 613), the trends, like a time series of results, are obtained and shown in step 614, as described above with FIG. 5. It should be appreciated that in another implementation the user is able to select which type of trends she/he is interested in, and then those trends are obtained and shown.

If the user does not request the trends (step 613), but selects to close the application, or close the application at any time, the application is closed in step 615.

It should be appreciated that in another implementation n-req subsequent frames may be inputted to the image processing and if one or more of them cannot be image processed, corresponding amount of frames is inputted to the image processing, etc. In a further implementation, subsequent frames are inputted to the image processing without waiting results until n-req results are obtained, and the possible additional results are simply ignored.

The above process may be implemented with the distributed tool as well, for example by performing the steps or part of the steps of FIG. 6 in the light tool unit. Further, the light tool unit may be configured to forward frames to the centralized analyzer tool until it receives a mean result to be shown to the user.

The accuracy of the image processing may be improved by processing additional comparison areas from the test as comparison areas. For example, square areas having a predetermined size and distance from the wells may be used as such additional comparison areas in the image processing. They can be used to fine tune the grey level determination, for example by applying fine tuning to grey shade results before step 713.

Although not illustrated in the above examples, it should be appreciated that if there are different tests for different purposes, the image processing unit may be configured to determine the purpose of the test from the received image, for example by means of some additional information, like a barcode, a type/purpose identifier, etc., or the user may have been prompted to select the purpose amongst shown options or to input some identification information of the test, or any other convenient way may be used for identifying the purpose of the test, the purpose being used for selecting statistical classifiers and a neural network trained for the purpose.

As is evident, the present invention is applicable to be used with any kind of test from which an image may be captured for image processing to perform an image processing to an image of the reaction results and the control in the test, outputs of which are analyzed and resulting results, such as test results, and/or conclusions based on the reaction results and/or the test results, then are shown to the user/consumer via a graphical user interface, thereby helping the user/consumer to detect the physiological status of the patient or pet.

Also a following method for determining a treatment for a subject in need thereof may be implemented:

a) contacting a sample obtained from the subject with a test for determining biomarkers,

b) allowing the sample to react in the test, and

c) capturing an image of the reaction results and the control in the test,

d) inputting the image to an image processing, the image processing outputting one or more test results,

e) determining one or more treatment suggestions on the basis of the one or more test results,

f) associating the test results with the one or more treatment suggestions; and

g) showing the test results and the one or more treatment suggestions via a graphical user interface.

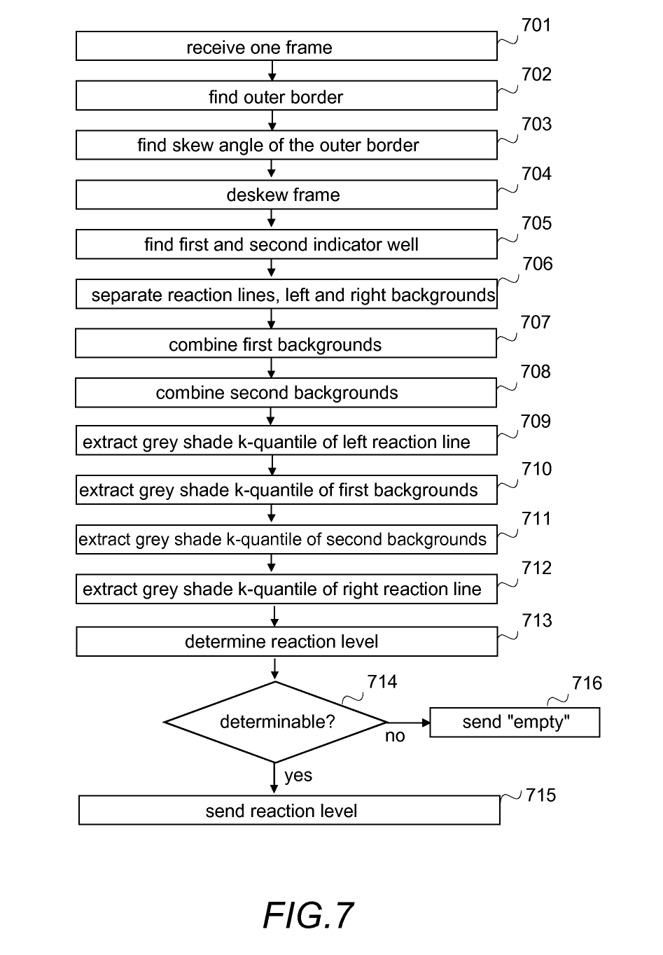

FIG. 8 is a simplified block diagram illustrating some units for an apparatus 800 configured to be an mobile device, i.e. an apparatus providing at least the camera unit and one of the tool units described above and/or one or more units configured to implement at least some of the functionalities described above with the mobile device. In the illustrated example the apparatus comprises one or more interfaces (IF) 801′ for receiving and transmitting communications, one or more user interfaces (U-IF) 801 for interaction with a user, a processor 802 configured to implement at least some functionality described above with a corresponding algorithm/algorithms 803 and a memory 804 usable for storing a program code required at least for the implemented functionality and the algorithms. For example, the algorithms may comprise for the stand-alone analyzer tool (tool unit, app) a trained statistical classifier for outer border finding, a trained statistical classifier for outer box finding and a trained neural network for reaction level determination, and a comparator to determine the result from the reaction level, updatable separately or together. If the tool unit is configured to store results, the memory 804 is usable for that purpose as well. Further, the memory 804 may be used also for storing the additional information or at least some pieces of the additional information.

FIG. 9 is a simplified block diagram illustrating some units for an apparatus 900 configured to be a server apparatus, i.e. an apparatus providing at least the image processing unit and/or one or more units configured to implement at least some of the functionalities described above with the server apparatus. In the illustrated example, the apparatus comprises one or more interfaces (IF) 901′ for receiving and transmitting information, a processor 902 configured to implement at least some functionality described above with a corresponding algorithm/algorithms 903, and memory 904 usable for storing a program code required at least for the implemented functionality and the algorithms. If the server apparatus is configured to store the results, the memory is used for that purpose, too.

In other words, an apparatus configured to provide the mobile device, and/or an apparatus configured to provide the server apparatus, or an apparatus configured to provide one or more corresponding functionalities, is a computing device that may be any apparatus or device or equipment configured to perform one or more of corresponding apparatus functionalities described with an embodiment/example/implementation, and it may be configured to perform functionalities from different embodiments/examples/implementations. The unit(s) described with an apparatus may be separate units, even located in another physical apparatus, the distributed physical apparatuses forming one logical apparatus providing the functionality, or integrated to another unit in the same apparatus.

The techniques described herein may be implemented by various means so that an apparatus implementing one or more functions of a corresponding apparatus described with an embodiment/example/implementation comprises not only prior art means, but also means for implementing the one or more functions of a corresponding apparatus described with an embodiment and it may comprise separate means for each separate function, or means may be configured to perform two or more functions. For example, the tool unit and/or the light tool unit and/or the image processing unit and/or algorithms, may be software and/or software-hardware and/or hardware and/or firmware components (recorded indelibly on a medium such as read-only-memory or embodied in hard-wired computer circuitry) or combinations thereof. Software codes may be stored in any suitable, processor/computer-readable data storage medium(s) or memory unit(s) or article(s) of manufacture and executed by one or more processors/computers, hardware (one or more apparatuses), firmware (one or more apparatuses), software (one or more modules),

An apparatus configured to provide the mobile device, and/or an apparatus configured to provide the server apparatus, and/or an apparatus configured to provide one or more corresponding functionalities, may generally include a processor, controller, control unit, micro-controller, or the like connected to a memory and to various interfaces of the apparatus. Generally the processor is a central processing unit, but the processor may be an additional operation processor. Each or some or one of the units and/or algorithms and/or calculation mechanisms described herein may be configured as a computer or a processor, or a microprocessor, such as a single-chip computer element, or as a chipset, including at least a memory for providing storage area used for arithmetic operation and an operation processor for executing the arithmetic operation. Each or some or one of the units and/or algorithms and/or calculation mechanisms described above may comprise one or more computer processors, application-specific integrated circuits (ASIC), digital signal processors (DSP), digital signal processing devices (DSPD), programmable logic devices (PLD), field-programmable gate arrays (FPGA), and/or other hardware components that have been programmed in such a way to carry out one or more functions or calculations of one or more embodiments. In other words, each or some or one of the units and/or the algorithms and/or the calculation mechanisms described above may be an element that comprises one or more arithmetic logic units, a number of special registers and control circuits.

Further, an apparatus implementing functionality or some functionality according to an embodiment/example/implementation of an apparatus configured to provide the mobile device, and/or an apparatus configured to provide the server apparatus, or an apparatus configured to provide one or more corresponding functionalities, may generally include volatile and/or non-volatile memory, for example EEPROM, ROM, PROM, RAM, DRAM, SRAM, double floating-gate field effect transistor, firmware, programmable logic, etc. and typically store content, data, or the like. The memory or memories may be of any type (different from each other), have any possible storage structure and, if required, being managed by any database management system. The memory may also store computer program code such as software applications (for example, for one or more of the units/algorithms/calculation mechanisms) or operating systems, information, data, content, or the like for the processor to perform steps associated with operation of the apparatus in accordance with examples/embodiments. The memory, or part of it, may be, for example, random access memory, a hard drive, or other fixed data memory or storage device implemented within the processor/apparatus or external to the processor/apparatus in which case it can be communicatively coupled to the processor/network node via various means as is known in the art. An example of an external memory includes a removable memory detachably connected to the apparatus.

An apparatus implementing functionality or some functionality according to an embodiment/example/implementation of an apparatus configured to provide the mobile device, and/or an apparatus configured to provide the server apparatus, or an apparatus configured to provide one or more corresponding functionalities, may generally comprise different interface units, such as one or more receiving units for receiving user data, control information, requests and responses, for example, and one or more sending units for sending user data, control information, responses and requests, for example. The receiving unit and the transmitting unit each provides an interface in an apparatus, the interface including a transmitter and/or a receiver or any other means for receiving and/or transmitting information, and performing necessary functions so that content and other user data, control information, etc. can be received and/or transmitted. The receiving and sending units may comprise a set of antennas, the number of which is not limited to any particular number.

Further, an apparatus implementing functionality or some functionality according to an embodiment/example/implementation of an apparatus configured to provide the mobile device, and/or an apparatus configured to provide the server apparatus, or an apparatus configured to provide one or more corresponding functionalities, may comprise other units.

The steps and related functions described above in FIGS. 4 and 5 are in no absolute chronological order, and some of the steps may be performed simultaneously or in an order differing from the given one. For example, extracting the mean grey levels may be performed simultaneously. Other functions can also be executed between the steps or within the steps. For example, if in the training material for the neural network the control line is always on the left and the reaction line in the right, a position of the test in the captured image (position corresponding to the training material or being inverted when compared with the training material) may be determined by using the grey level values in solutions in which the control line is always darker than the reaction line, or if the test contains the bar code or some other additional information, it may be used for determining the position, the determination of the position being used to input determined mean grey levels to the neural network properly, for example. Some of the steps or part of the steps can also be left out or replaced by a corresponding step or part of the step.

It will be obvious to a person skilled in the art that, as the technology advances, the inventive concept can be implemented in various ways. The invention and its embodiments are not limited to the examples described above but may vary within the scope of the claims.

### EXAMPLES

### 1. Principle of the Test in Brief

The theory of principle and practical aspects in the manufacturing of a POC-test strip are presented and discussed in detail for example in the following articles. The methods described in these articles or any other documents related to POC test strips can be utilized by a man skilled in the art for producing a test strip for the present invention.
1. Leuvering J H W, Thal P J H M, van der Waart M, Schuurs A H. J Immunoassay Immunochem (1980) 1:77-91.
2. Leuvering J H W, Thal P J H M, van der Vaart M, Schuurs A H. J Immunol Methods (1981) 45:183-194.
3. van Amerongen A, Wichers J H, Berendsen L, Timmermans A J M, Keizer G D, van Doorn A W J, Bantjes A, van Gelder W M J. J Biotechnol (1993) 30:185-195.
4. Osikowicz G, Beggs M, Brookhart P, Caplan D, Ching S F, Eck P, et al. Clin Chem (1990) 36:1586.
5. Posthuma-Trumpie G, Korf J, van Amerongen A. Anal Bioanal Chem (2009) 393:569-582.

### 2. Description of the Production of the Test Strip

The test device comprises the following parts or materials
1. biological materials, which include one or two antibodies, and possibly a labeled competing analyte depending of the assay type
2. auxiliary, inactive materials to
   a. aid in the application of antibodies onto the membrane
   b. aid in labeling the antibodies or analytes with the particles
3. (gold, latex, magnetic or fluorescent) particles used in the labeling of the secondary antibody or the competing analyte
4. a sample pad, made of polyester or equivalent material, where the sample is placed
5. a conjugate pad, on which the labeled material is dispensed
6. a nitrocellulose or equivalent membrane on which the appropriate capture antibodies are immobilized, and in which the sample migrates towards the reaction zone (e.g. test and control lines)
7. supportive (plastic) cassette with sample applying port and reading frame for the prepared membrane

### The Production of the Test Device in Detail

### Production and Immobilization of Labeled Particles

The conjugate pad, made from polyester, was immersed in a solution containing 0.5% of sucrose, 1% of bovine serum albumin (BSA), 10% Tris buffer in water for 1 h, and then dried at RT for 1 h.

Gold particles were labeled with anti-cortisol-antibody. Diluted antibody was added to gold sol by mixing at the same time. 10% BSA-solution was added after mixing, and the total solution was mixed again. Gold particles were centrifuged until a clear supernatant was achieved, after which supernatant was replaced with diluted BSA solution, sonicated and centrifugated again. The supernatant was then again replaced with glycine buffer containing BSA and sucrose.

Anti-cortisol-gold particles were applied to the conjugate pad by a dispenser system in an amount of approximately 10 μl/cm. The applied conjugate pad was dried with a fan and stored in a dry state until use.

### Production and Assembly of the Test Device

The test strip comprised of 4 main elements: a sample pad, a conjugate pad, a nitrocellulose membrane, and an absorbent pad. The strip was positioned inside the plastic cassette in such a way that the ends of the elements overlapped, ensuring a continuous flow by capillary action of the developing solution from the sample pad to the absorbent pad.

0.5 g/l anti-cortisol capture antibody (test line) and goat anti-mouse antibody were applied to the membrane by a dedicated dispenser system. After immobilization both antibody lines were dried, and membrane was washed with blocking solution containing 0.5% mannitol, 0.25% BSA, 0.05% Tween in water. The membrane was dried at RT, and stored in a dry state until use.

### 3. Use of a Test Strip for Biological Samples

Any human or animal biological sample such as a urine, feces, breathing, brush or saliva sample, a tissue fragment, a secretion sample or a blood sample (eg. whole blood, serum or plasma), preferably a urine sample, was applied to a test strip. The sample was allowed to react in the test. A mobile device was used to take a video of the test strip in order to receive the test results.

### 4. Comparison Studies

A comparison study was carried out with the method of the present invention as described above in the Example chapter (Evice™ lateral flow test). A mobile was used to take a short video of the test strip in order to receive the test results. In the comparison method analyzer Siemens Immulite 2000 was utilized. Canine urine cortisol concentrations (nmol/l) were detected by both methods. The results are shown in table 1.

TABLE 1
Results of the reference method and the present invention
(i.e. Evice ™ lateral flow test with mobile
application read-out) using parallel urine samples (n)

| | Siemens Im- | | |
|---|---|---|---|
| Mean cortisol (nmol/l) | mulite 2000 | Evice | |
| Siemens Immulite 2000 | (SD) | (SD) | n |
| 256 | +/−25.3 | +/−26.5 | 5 |
| 598 | +/−46.1 | +/−47.4 | 5 |
| 920 | +/−68.7 | +/−64.6 | 5 |

When teaching the neural network in the calibration mode of the application, first multiple parallel urine samples were run and the concentration of cortisol was measured in the lab using Siemens Immulite 2000 immunochemistry analyzer (reference method giving the concentrations of cortisol, for example 598 nmol/l (mean of 5 parallel samples), 256 nmol/l and 920 nmol/l).

Then the neural network was taught and the tool unit (app) was calibrated. The parallel urine samples of mean concentrations of 598, 256 and 920 nmol/l were then pipetted into the test and, when in the calibration mode, the concentration was given to the app.

During the time interval of 15-45 minutes from pipetting tens of thousands of scans with different phone models (e.g. iPhone4, iPhone5, different Android phones) were performed and stored under different lightning conditions.

1. A test method for determining a result based on the presence, absence or concentration of a biomarker in a sample of a subject, wherein the method comprises:
a) contacting a sample obtained from a subject with a biomarker test for determining a biomarker or biomarkers,
b) allowing the sample to react in the biomarker test,
c) providing only the biomarker of step b) and a mobile device and capturing by said mobile device, which is either a smart phone or a tablet, at least one image of the reaction results and the control in the biomarker test,
d) inputting the at least one image to an image processing of said mobile device, the image processing outputting one or more test results indicating the presence, absence or concentration of the biomarker in the sample, and
e) showing the test results and/or a conclusion drawn from the test results by said mobile device via a graphical user interface of said mobile device.

2-15. (canceled)

16. The method of claim 1, wherein the biomarker test comprises an antibody based assay.

17. The method of claim 1, wherein the biomarker test is a lateral flow assay.

18. The method of claim 1, wherein the biomarker is selected from a group consisting of cortisol, RBP (Retinol Binding Protein), bile acids, progesterone, BNP (B-type Natriuretic Peptide or Brain-derived Natriuretic Peptide), proBNP, NT-proBNP, troponin I (TnI), troponin T (TnT), DHEA (DiHydroEpi-Androsteron), DHEA-S (DiHydroEpiAndrosteroni-Sulphate), PSA (Prostata Specific Antigen), PAP (Prostatic Acid Phosphatase), trypsinogen, myoglobin, rheumatoid factor, cyclic citrullinated peptide, neopterin, catecholamines, de-oxypyridinoline, N-telopeptide (NTX), and beta-2-microglobulin.

19. The method of claim 1, wherein the sample is a blood, saliva, feces or a urine sample.

20. The method of claim 1, wherein the subject is a human or an animal.

21. The method of claim 1, wherein the animal is selected from a group consisting of a canine, feline, equine, pig, ruminant, camelid or zoo animal.

22. The method of claim 1, wherein the biomarker test is an antibody based biomarker test for an animal, determining aberrant cortisol concentration in a urine sample obtained from the animal.

23. The method of claim 1, wherein the image processing carried out by the mobile device comprises for an image:
finding from the image an outer border of the test,
finding within the outer border a first and a second indicator well area,
separating from each well area a reaction line and a left background area and a right background area;
extracting a mean grey level of the reaction line in the first indicator well area,
extracting a mean grey level of the left background areas of the first well and the second well,
extracting a mean grey level of the right background areas of the first well and the second well,
extracting a mean grey level of the reaction line in the second indicator well area, and
calculating by a neural network one or more reaction levels using the mean grey levels as inputs for the neural network.

24. The method of claim 1, wherein the image processing carried out by the mobile device comprises for an image:
finding from the image an outer border of the test,
finding within the outer border a first and a second indicator well area,
separating from the first well area a first line, a first left background area and a first right background area;
combining the first left background area and the first right back-ground area as a first combined area;
separating from the second well area a second line, a second left background area and a second right background area;
combining the second left background area and the second right background area as a second combined area;
determining statistical information of grey levels from the first line, from the second line, from the first combined area and from the second com-bined area; and
using the determined grey levels as inputs for the neural network.

25. A test arrangement determining the presence, absence or concentration of a biomarker in a sample of a subject, consisting of
a) a biomarker test for determining biomarkers, and
b) a mobile device, which is either a smart phone or a tablet, configured to determine the presence, absence or concentration of a biomarker in a sample of a subject from one or more images of the used biomarker test.

26. The test arrangement of claim 25 wherein the biomarker test comprises an antibody based assay.

27. The test arrangement of claim 25, wherein the biomarker test is a lateral flow assay.

28. The test arrangement of claim 25, wherein the biomarker is selected from a group consisting of cortisol, RBP (Retinol Binding Protein), bile acids, progesterone, BNP (B-type Natriuretic Peptide or Brain-derived Natriuretic Peptide), proBNP, NT-proBNP, troponin I (TnI), troponin T (TnT), DHEA (DiHydroEpi-Androsteron), DHEA-S (DiHydroEpiAndrosteroni-Sulphate), PSA (Prostata Specific Antigen), PAP (Prostatic Acid Phosphatase), trypsinogen, myoglobin, rheumatoid factor, cyclic citrullinated peptide, neopterin, catecholamines, de-oxypyridinoline, N-telopeptide (NTX), and beta-2-microglobulin.

29. The test arrangement of claim 25, wherein the subject is a human or an animal.

30. The test arrangement of claim 25, wherein the animal is selected from a group consisting of a canine, feline, equine, pig, ruminant, camelid or zoo animal.

31. The test arrangement of claim 25, wherein the biomarker test is an antibody based biomarker test for an animal, determining aberrant cortisol concentration in a urine sample obtained from the animal.

32. The test arrangement of claim 25, wherein the image processing carried out by the mobile device comprises for an image:
finding from the image an outer border of the test,
finding within the outer border a first and a second indicator well area,
separating from each well area a reaction line and a left background area and a right background area;
extracting a mean grey level of the reaction line in the first indicator well area,
extracting a mean grey level of the left background areas of the first well and the second well,
extracting a mean grey level of the right background areas of the first well and the second well,
extracting a mean grey level of the reaction line in the second indicator well area, and
calculating by a neural network one or more reaction levels using the mean grey levels as inputs for the neural network.

33. The test arrangement of claim 32, wherein the image processing carried out by the mobile device comprises for an image:
finding from the image an outer border of the test,
finding within the outer border a first and a second indicator well area,
separating from the first well area a first line, a first left background area and a first right background area;
combining the first left background area and the first right background area as a first combined area;
separating from the second well area a second line, a second left background area and a second right background area;
combining the second left background area and the second right background area as a second combined area;
determining statistical information of grey levels from the first line, from the second line, from the first combined area and from the second combined area; and
using the determined grey levels as inputs for the neural network.

34. A mobile device, which is either a smart phone or a tablet, comprising:
at least one user interface;
at least one camera unit;
at least one processor and at least one memory including a computer program code, wherein the at least one memory and the computer program code are configured, with the at least one processor, to cause the mobile device to implement at least an analyzer tool loaded into the mobile device and to perform, in response to detecting that the analyzer tool is selected via the user interface, operations comprising:
activating the at least one camera unit for taking one or more images;
inputting the one or more images to an image processing of the analyzer tool, the image processing being configured to determine image by image from one image a grey level of a first background area in a test, a grey level of a second background area in the test, a grey level of a first line splitting the first background area and a grey level of a second line splitting the second background area;
inputting the grey levels obtained as output from the image processing to a trained neural network of the analyzer tool, the neural network being trained to output the presence, absence or concentration of a biomarker;
outputting via the user interface the output of the trained neural network and/or a conclusion determined from the output of the trained neural network.

35. A mobile device of claim 34, wherein the at least one memory and the computer program code are configured, with the at least one processor, to cause the mobile device to perform further operations comprising:
monitoring that a predetermined number of outputs are received from the trained neural network;
deactivating, in response to the predetermined number of outputs being received from the trained neural network, the camera unit;
calculating, in response to the predetermined number of outputs being received from the trained neural network, a statistical value from the outputs; and
outputting via the user interface the calculated statistical value and/or a conclusion determined from statistical value.

36. A mobile device of claim 34, wherein the at least one memory and the computer program code are configured, with the at least one processor, to cause the mobile device to perform the image processing according to the method of claim 9.
